(19) United States
(12) Patent Application Publication
(10) Pub. No.: US 2025/0027054 A1
(43) Pub. Date: Jan. 23, 2025

(54) Compositions And Methods For Differentiation Of Human Pluripotent Stem Cells Into Desired Cell Types

(71) Applicant: Elixirgen Scientific, Inc, Baltimore, MD (US)

(72) Inventor: Minoru S.H. Ko, Baltimore, MD (US)

(73) Assignee: Elixirgen Scientific, Inc, Baltimore, MD (US)

(21) Appl. No.: 18/786,913

(22) Filed: Jul. 29, 2024

Related U.S. Application Data
(62) Division of application No. 17/458,609, filed on Aug. 27, 2021, now Pat. No. 12,098,392.
Application No. 17/458,609 is a division of application No. 15/905,982, filed on Feb. 27, 2018, now Pat. No. 11,136,555.
(60) Provisional application No. 62/523,324, filed on Jun. 22, 2017.
(60) Provisional application No. 62/465,188, filed on Mar. 1, 2017.

(51) Int. Cl. C12N 5/074; C07K 14/00; C12N 5/071; C12N 5/077; C12N 5/0789; C12N 5/0793; C12N 15/113; C12N 15/85; C12N 15/86

(52) U.S. Cl. CPC C12N 5/0696; C07K 14/00; C12N 5/0619; C12N 5/0647; C12N 5/0658; C12N 5/067; C12N 15/113; C12N 15/85; C12N 15/86; C12N 2015/8518; C12N 2310/14; C12N 2501/60; C12N 2501/602; C12N 2501/603; C12N 2506/02; C12N 2506/03; C12N 2506/08; C12N 2506/1323; C12N 2506/14; C12N 2506/45; C12N 2510/00; C12N 2523/00; C12N 2760/18741; C12N 2760/18843; C12N 2800/107

(57) ABSTRACT

In related-art methods of differentiating pluripotent stem cells into a desired cell type, there has not been established a differentiation induction method using human ES/iPS cells and being stable and highly efficient. The use of complicated culture steps is a large problem. In addition, there are also large problems in, for example, that the speed of cell differentiation is low, and hence long-period culture is required, and that the differentiation efficiency is low, and hence it is difficult to obtain a sufficient number of required cells. A method of inducing differentiation into a desired cell type, which induces differentiation within a short period of time and with high efficiency by the use of a Sendai virus vector capable of expressing a transcription factor, and as required, the use of a pluripotent stem cell in which an expression amount of a POU5F1 protein has been substantially removed or reduced, is provided.

### BACKGROUND OF THE INVENTION

### 1. Field of the Invention

The present application is a divisional application of U.S. patent application Ser. No. 17/458,609 filed Aug. 27, 2021, which in turn is a divisional application of U.S. patent application Ser. No. 15/905,982 filed Feb. 27, 2018 which claims priority from U.S. Provisional Patent Application No. 62/465,188 and U.S. Provisional Patent Application No. 62/523,324, which are incorporated herein by reference. The present invention relates to a method of efficiently differentiating a pluripotent stem cell into a desired cell type.

### REFERENCE TO AN ELECTRONIC SEQUENCE LISTING

The contents of the electronic sequence listing (3190_219_03-Sequence_Listing-revised.xml; Size: 36,734 bytes; and Date of Creation: Oct. 14, 2024) is herein incorporated by reference in its entirety.

### 2. Description of the Related Art

Human pluripotent stem cells (hPSCs), such as human embryonic stem cells (hESCs) and human induced pluripotent stem cells (hiPSCs), have potentials to become all cell types in a human body in vitro. For the last 20 years, use of neurons, muscles, and other cell types differentiated from hPSCs for cell transplantation therapy and drug screening has been widely investigated, and practical application thereof has been under consideration. However, the practical application is limited, and one of the major obstacles is difficulty of differentiating hPSCs into desired cell types, such as neurons and muscles.

The most generally used method is step-by-step differentiation based on successive changes in cell culture environment (Hu et al., 2010, PNAS 107, 4335-4340). Those differentiation steps have been well established and widely used, but have fundamental limitations in both speed and scale. Further, the process is relatively slow, and takes several weeks until formation of functional neurons as the desired cell type.

As another method, PSCs can be rapidly and efficiently differentiated by inducing or overexpressing transcription factors (TFs) with plasmids, viruses, and other vectors (Busskamp et al., 2014, Molecular Systems Biology 10, 760; Hester et al., Molecular Therapy, 19, 1905-1912; Zhang et al., 2013, Neuron, 78, 785-798).

The inventors of the present invention have reported rapid and efficient differentiation into skeletal muscles (Akiyama et al., 2016, Development 143, 3674), motor neurons (Goparaju et al., 2017, Scientific Reports 7, 42367), and lacrimal gland epithelium-like cells (Hirayama et al., npj Aging and Mechanisms of Disease 2017, 1) by transfection of a cocktail of synthetic mRNAs encoding TFs into hPSCs. The differentiation method based on synthetic mRNA has many desirable features. mRNA is not incorporated into a cellular genome, and hence achieves safe and footprint-free transfer of a gene product. However, this method requires a plurality of times of transfection of synthetic mRNAs into cells, typically one or two times of transfection a day for several consecutive days. Thus, this method puts a huge burden on an experimenter, and requires expert skills.

In related-art methods of differentiating pluripotent stem cells into a desired cell type, there has not been established a differentiation induction method using human ES/iPS cells and being stable and highly efficient. Many attempts have been made, which includes a step-by-step differentiation induction method based on the control of culture conditions or the addition of, for example, various cell growth factors/differentiation factors to a culture solution, but the use of complicated culture steps is a big problem. In addition, there are also big problems in, for example, that the speed of cell differentiation is low, and hence long-period culture is required, and that the differentiation efficiency is low, and hence it is difficult to obtain a sufficient number of required cells.

### SUMMARY OF THE INVENTION

The inventors of the present invention have developed a method of inducing differentiation into a desired cell type within a short period of time and with high efficiency by the use of a Sendai virus vector capable of expressing a transcription factor, and as required, the use of a pluripotent stem cell in which an expression amount of a POU5F1 protein has been substantially removed or reduced.

Thus, the present invention has been completed.

That is, the present invention includes the following.
1. A method of differentiating a pluripotent stem cell into a desired cell type, including:
   1) adding a Sendai virus vector containing a transcription factor required for induction of differentiation into the desired cell type to a pluripotent stem cell in a cell culture medium; and
   2) culturing the pluripotent stem cell to differentiate the pluripotent stem cell into the desired cell type.
2. A method according to the above-mentioned item 1, further including adding a gene for a compound having an action of substantially removing or reducing an expression amount of a POU5F1 protein to the pluripotent stem cell.
3. A method according to the above-mentioned item 2, wherein the gene for the compound having an action of substantially removing or reducing an expression amount of a POU5F1 protein includes siRNA against POU5F1.
4. A method according to the above-mentioned item 1, in which the pluripotent stem cell includes a pluripotent stem cell in which an expression amount of a POU5F1 protein has been substantially removed or reduced.
5. A method according to any one of the above-mentioned items 1 to 4, wherein the adding a Sendai virus vector containing a transcription factor required for induction of differentiation into the desired cell type is performed once.
6. A method according to any one of the above-mentioned items 1 to 5, wherein the Sendai virus vector is temperature-sensitive.
7. A method according to any one of the above-mentioned items 1 to 6, wherein the desired cell type includes a skeletal muscle cell, and wherein the transcription factor includes MYOD1.
8. A method according to the above-mentioned item 7, wherein differentiation efficiency of the skeletal muscle cell is 75% or more.
9. A method according to the above-mentioned item 7 or 8, wherein differentiation efficiency of the skeletal muscle cell is 75% or more, and wherein one kind of cell culture medium is used.
10. A method according to any one of the above-mentioned items 1 to 6, wherein the desired cell type includes a motor neuron, and wherein the transcription factor includes NEUROG3.
11. A method according to the above-mentioned item 10, wherein differentiation efficiency of the motor neuron is about 90%.
12. A method according to the above-mentioned item 10 or 11, wherein differentiation efficiency of the motor neuron is about 90%, and wherein one kind of cell culture medium is used.
13. A method according to any one of the above-mentioned items 1 to 6, wherein the desired cell type includes a liver cell, and wherein the transcription factor includes FOXA1 and HNF1A.
14. A method according to the above-mentioned item 13, wherein two kinds of cell culture media are used.
15. A method according to any one of the above-mentioned items 1 to 6, wherein the desired cell type includes a hematopoietic cell, and wherein the transcription factor includes SPI1.
16. A method according to any one of the above-mentioned items 1 to 6, wherein the desired cell type includes a dopaminergic neuron, and wherein the transcription factor includes FOXA1.

The method of efficiently differentiating a pluripotent stem cell into a desired cell type of the present invention has at least any one or more of the following effects.
(1) The period of time required for cell differentiation starting with the pluripotent stem cell is shortened and the differentiation induction efficiency is improved.
(2) Differentiation can be performed by adding the transcription factor required for induction of differentiation into the desired cell type only once.
(3) The number of kinds of transcription factors required for induction of differentiation into the desired cell type can be decreased as compared to related-art methods.
(4) When the method is combined with a method of reducing the undifferentiation maintenance of a pluripotent stem cell and/or a method of reducing the differentiation resistance thereof, the period of time required for cell differentiation starting with the pluripotent stem cell is shortened and the differentiation induction efficiency is improved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, and FIG. 1C are a schematic and images for illustrating and showing differentiation into skeletal muscles. FIG. 1A is a schematic of typical experimental steps. On Day 1, human iPS cells were plated on a cell culture dish. Immediately after the plating, a Sendai virus vector encoding a human MYOD1 gene was added to the cell culture medium. The cells were cultured in a CO₂ incubator at 33° C. On Day 2, siPOU5F1 was added to the cell culture medium. On Day 4, the temperature of the CO₂ incubator was changed to 37° C. On Day 6, in order to evaluate the efficiency of cell differentiation, the cells were fixed, and used for immunostaining. FIG. 1B is a microscopic image (10× objective lens) of cells immunostained with anti-myosin heavy chain (red signal), which is specific for mature skeletal muscles. The cells were further stained with DAPI (green signal) for visualizing the nuclei of all cells. FIG. 1C is a microscopic image (20× objective lens) of cells immunostained with anti-myosin heavy chain (red signal), which is specific for mature skeletal muscles. The cells were further stained with DAPI (green signal) for visualizing the nuclei of all cells.

FIG. 2A, FIG. 2B, FIG. 2C, FIG. 2D, and FIG. 2E are a schematic and images for illustrating and showing differentiation into cholinergic and/or motor neurons. FIG. 2A is a schematic of typical experimental steps. On Day 1, human iPS cells were plated on a cell culture dish. Immediately after the plating, a Sendai virus vector encoding a human NGN3 gene was added to the cell culture medium. The cells were cultured in a CO₂ incubator at 33° C. On Day 3, the temperature of the CO₂ incubator was changed to 37° C. On Day 4, the cells were re-plated on a glass coverslip. On Day 6, in order to evaluate the efficiency of cell differentiation, the cells were fixed, and used for immunostaining. FIG. 2B is a microscopic image (20× objective lens) of cells stained with DAPI (blue signal) for visualizing the nuclei of all cells. FIG. 2C is a microscopic image (20× objective lens) of cells immunostained with anti-β3-tubulin (TUBB3) (red signal), which is specific for mature neurons. FIG. 2D is a microscopic image (20× objective lens) of cells immunostained with anti-choline acetyltransferase (ChAT) antibody (green signal), which is specific for mature motor neurons. FIG. 2E is a synthetic image of FIG. 2C and FIG. 2D.

FIG. 3A, FIG. 3B, FIG. 3C, FIG. 3D, and FIG. 3E are a schematic and images for illustrating and showing differentiation into liver cells. FIG. 3A is a schematic of typical experimental steps. On Day 1, human iPS cells were plated on a cell culture dish. Immediately after the plating, a mixture of equal amounts of a Sendai virus vector encoding a human FOXA1 gene and a Sendai virus vector encoding a human HNF1A gene was added to the cell culture medium. The cell culture medium is a ROCK-inhibitor (Y27632)-containing StemFit (registered trademark in Japan) Basic02 (Ajinomoto). The cells were cultured in a CO₂ incubator at 33° C. On Day 2, siPOU5F1 was added to the cell culture medium. The cell culture medium was replaced with a differentiation medium. On Day 5, the temperature of the CO₂ incubator was changed to 37° C. On Day 7, the cell culture medium was replaced with a maturation medium. On Day 8, in order to evaluate the efficiency of cell differentiation, the cells were fixed, and used for immunostaining. FIG. 3B is a microscopic image (10× objective lens) of cells immunostained with anti-α-fetoprotein (AFP) antibody (red signal), which is a fetal form of albumin and is specific for embryonic liver cells. FIG. 3C is a microscopic image (10× objective lens) of cells immunostained with anti-albumin (ALB) antibody (green signal), which is specific for liver cells. FIG. 3D is a microscopic image (20× objective lens) of cells immunostained with anti-α-fetoprotein (AFP) antibody (red signal), which is a fetal form of albumin and is specific for embryonic liver cells. FIG. 3E is a microscopic image (20× objective lens) of cells immunostained with anti-albumin (ALB) antibody (green signal), which is specific for liver cells.

### DESCRIPTION OF THE EMBODIMENTS

### The Present Invention

A method of efficiently differentiating a pluripotent stem cell into a desired cell type of the present invention (hereinafter sometimes referred to as “method of the present invention”) includes part or all of the following steps, though the method is not particularly limited as long as the method uses a Sendai virus vector capable of expressing a transcription factor required for induction of differentiation into the desired cell type:
1) a step of adding a Sendai virus vector containing a transcription factor required for induction of differentiation into the desired cell type to a pluripotent stem cell in a cell culture medium; and
2) a step of culturing the pluripotent stem cell to differentiate the pluripotent stem cell into the desired cell type.

The adding of various compounds, transcription factors, vectors and the like to the pluripotent stem cell encompasses addition to a medium in which the pluripotent stem cell is present.

### Pluripotent Stem Cell

The pluripotent stem cell to be used in the method of the present invention is not particularly limited, but is preferably derived from a mammal, particularly preferably derived from a human. The pluripotent stem cell is, for example, a human ES cell, a human iPS cell, or any combination thereof, is not particularly limited, and encompasses tissue stem cells derived from tissues and organs, dermal fibroblasts, and all kinds of cells derived from tissues and organs.

### Sendai Virus Vector

Sendai virus is a kind of virus of the genus Respirovirus of the family Paramyxoviridae, and has single-stranded RNA as its genes.

The Sendai virus vector to be used in the method of the present invention may be a natural strain, a wild-type strain, a mutant strain, or a commercially available product (e.g., from ID Pharma).

An example thereof may be an F gene-deleted Sendai virus vector having G69E, T116A, and A183S mutations in M protein, A262T, G264R, and K461G mutations in HN protein, D433A, R434A, K437A, and L511F mutations in P protein, and L1361C, L1558I, N1197S, and K1795E mutations in L protein.

In addition, the Sendai virus vector to be used in the method of the present invention is preferably a temperature-sensitive strain. The term “temperature-sensitive” refers to a significant lowering of activity at a general cell culture temperature (e.g., from 37° C. to 38° C.) as compared to low temperature (e.g., from 30° C. to 36° C.). For example, mutations such as TS 7 (Y942H/L1361C/L1558I mutations in the L protein), TS 12 (D433A/R434A/K437A mutations in the P protein), TS 13 (D433A/R434A/K437A mutations in the P protein and an L1558I mutation in the L protein), TS 14 (D433A/R434A/K437A mutations in the P protein and an L1361C mutation in the L protein), and TS 15 (D433A/R434A/K437A mutations in the P protein and L1361C/L1558I mutations in the L protein) of the Sendai virus are temperature-sensitive mutations, and may be suitably utilized in the present invention.

For those Sendai virus vectors, reference may be made to Japanese Patent Re-publication No. 2015/046229.

### Transcription Factor Required for Induction of Differentiation into Desired Cell Type

The form of the “transcription factor required for induction of differentiation into the desired cell type” to be used in the method of the present invention is not particularly limited as long as the transcription factor can be carried on the Sendai virus vector, but examples thereof may include, but not particularly limited to, nucleic acids, such as RNA and DNA, and synthetic nucleic acids.

In addition, in the method of the present invention, examples of the desired cell type may include skeletal muscles (skeletal muscle cells), nerve cells (motor neurons and dopaminergic neurons), the liver (liver cells), chondrocytes, bone cells, and hematopoietic cells.

As described in Examples below, in the method of the present invention, differentiation can be performed by adding the transcription factor required for induction of differentiation into the desired cell type only once.

Further, as described in Examples below, in the method of the present invention, the number of kinds of required transcription factors has been successfully reduced as compared to related-art methods by carrying the transcription factor required for induction of differentiation into the desired cell type on the Sendai virus vector.

In addition, as described in Examples below, in the method of the present invention, the differentiation efficiency has been successfully improved as compared to related-art methods by carrying the transcription factor required for induction of differentiation into the desired cell type on the Sendai virus vector.

Besides, as described in Examples below, in the method of the present invention, the number of kinds of required transcription factors has been successfully reduced and the differentiation efficiency has been successfully improved as compared to related-art methods by: carrying the transcription factor required for induction of differentiation into the desired cell type on the Sendai virus vector; and adding a gene for a compound having an action of substantially removing or reducing an expression amount of a POU5F1 protein to the pluripotent stem cell.

### Method of Efficiently Inducing Differentiation of Pluripotent Stem Cell into Desired Cell Type of the Present Invention

In the method of the present invention, a method of reducing the undifferentiation maintenance of a pluripotent stem cell, and as required, a method capable of reducing the differentiation resistance of a pluripotent stem cell to the desired cell type may be preferably introduced. Examples thereof may include the following.

### Pluripotent Stem Cell Whose Undifferentiation Maintenance has been Reduced

In pluripotent stem cells, the expression of a transcription factor POU5F1 (SEQ ID NOS: 1 and 2: POU domain, class 5, transcription factor 1 isoform 1: ncbi.nlm.nih.gov/protein/NP_002692, other names: OCT3, OCT4, OTF3, OTF4, OTF-3, Oct-3, Oct-4, MGC22487) is essential to the undifferentiation maintenance of the pluripotent stem cells. POU5F1 is specifically expressed in pluripotent cells, such as reproductive cells and a preimplantation early embryo. That is, the “reducing the undifferentiation maintenance of a pluripotent stem cell” in the present invention means substantially removing or reducing an expression amount of a POU5F1 protein in the pluripotent stem cell. The substantially removing or reducing an expression amount of a POU5F1 protein encompasses inhibiting the process of any one of the transcription and translation stages of POU5S1 and/or inhibiting the activity of the translated POU5F1 protein, and is not particularly limited.

Besides, a state in which the expression amount of the POU5F1 protein in the pluripotent stem cell has been substantially removed or reduced may be confirmed by a comparison to the degree of the expression amount of the POU5S1 protein (or expression amount of the POU5F1 gene) in a pluripotent stem cell that has not been subjected to the removing or the reducing. For example, the state (degree) in which the expression amount of the POU5F1 protein in the pluripotent stem cell has been substantially removed or reduced is from 95 to 1, from 90 to 2, from 85 to 3, from 80 to 4, from 75 to 5, from 70 to 6, from 65 to 7, from 60 to 8, from 50 to 10, from 40 to 15, from 30 to 20, or about 25 when compared to the expression amount of the POU5F1 protein in the pluripotent stem cell that has not been removed or reduced, which is defined as 100. The degree of the expression amount of the POU5F1 protein in the pluripotent stem cell may be easily measured by using a commercially available anti-POU5F1 antibody, and the gene expression amount of POU5F1 may be measured by a method known per se (see WO 2017/131238 A1).

### Reducing Differentiation Resistance of Pluripotent Stem Cell to Desired Cell Type

In pluripotent stem cells, a special chromatin structure called a “bivalent domain” is formed in each promoter region of a group of genes involved in differentiation, and under a stemness-maintaining state, the group of genes involved in development/differentiation are in a standby state so as not to be easily expressed. The inventors of the present invention have confirmed that “when a methyl group modification of a histone called H3K27me3 is removed or reduced in the “bivalent domain”, the expression of differentiation genes required for induction of differentiation into the desired cell type is rapidly and efficiently facilitated” (see WO 2017/073763 A1).

That is, the “reducing differentiation resistance of a pluripotent stem cell to a desired cell type” of the present invention means that the H3K27me3 modification of the pluripotent stem cell is substantially removed or reduced.

In addition, a state in which the H3K27me3 modification of the pluripotent stem cell has been substantially removed or reduced may be confirmed by a comparison to the degree of the H3K27me3 modification of a pluripotent stem cell that has not been subjected to the removing or the reducing. For example, the state (degree) in which the H3K27me3 modification of the pluripotent stem cell has been substantially removed or reduced is from 95 to 1, from 90 to 2, from 85 to 3, from 80 to 4, from 75 to 5, from 70 to 6, from 65 to 7, from 60 to 8, from 50 to 10, from 40 to 20, or about 30 when compared to the degree of the H3K27me3 modification of the pluripotent stem cell that has not been removed or reduced, which is defined as 100. The degree of the H3K27me3 modification of the pluripotent stem cell may be easily measured by using a commercially available anti-Histone H3K27me3 antibody, and the gene expression amount of H3K27me3 may be measured by a method known per se.

### Use of Modified Synthetic mRNA for Target Gene

The method of the present invention preferably includes adding (introducing), to the pluripotent stem cell, a gene for a compound having an action of substantially removing or reducing an expression amount of a POU5F1 protein (a gene expressing small interfering RNA (siRNA) against POU5F1, a gene expressing shRNA against POU5F1, a gene expressing an antisense strand of POU5F1, or an antibody gene).

Similarly, the method of the present invention preferably includes adding (introducing), to the pluripotent stem cell, a gene for a compound having an action of substantially removing or reducing H3K27me3 modification.

The term “gene” as used herein encompasses not only double-stranded nucleic acids, but also their respective constituent single strands, such as plus strands (or sense strands) or complementary strands (or antisense strands), linear nucleic acids, and circular nucleic acids, and encompasses DNA, RNA, mRNA, cDNA, and the like, unless otherwise stated.

Besides, the target gene is meant to encompass the gene for the compound having an action of substantially removing or reducing an expression amount of a POU5F1 protein, and/or the gene for the compound having an action of substantially removing or reducing H3K27me3 modification, and the transcription factor required for induction of differentiation into the desired cell type.

A method known per se may be used without any particular limitation as a method of adding (introducing) the gene for the compound having an action of substantially removing or reducing an expression amount of a POU5F1 protein or the gene for the compound having an action of substantially removing or reducing H3K27me3 modification to the pluripotent stem cell. There is preferably used a method of inducing differentiation by efficiently introducing synthetic mRNA into human pluripotent stem cells through use of a gene expression method involving using synthetic mRNA developed by Warren, Rossi, et al. (reference: Cell Stem Cell 7:618-630, 2010.), which is a footprint-free forced gene expression method causing no gene incorporation into a host genome (see WO 2017/131238 A1).

The timing, at which the gene for the compound having an action of substantially removing or reducing an expression amount of a POU5F1 protein (or the gene for the compound having an action of substantially removing or reducing H3K27me3 modification) and the Sendai virus vector containing the transcription factor required for induction of differentiation into the desired cell type are added to the pluripotent stem cell, is not particularly limited. It is preferred that the gene for the compound having an action of substantially removing or reducing an expression amount of a POU5F1 protein (or the gene for the compound having an action of substantially removing or reducing H3K27me3 modification) be added to the pluripotent stem cell after the addition of the Sendai virus vector containing the transcription factor required for induction of differentiation into the desired cell type.

Further, the timing and number of times of the addition of each gene (mRNA) are not particularly limited. In the method of the present invention, unlike the related art, differentiation can be performed with high efficiency by adding the transcription factor required for induction of differentiation into the desired cell type (Sendai virus vector containing the transcription factor required for induction of differentiation into the desired cell type), and as required, the gene for the compound having an action of substantially removing or reducing an expression amount of a POU5F1 protein once during culture.

### Synthesis of Modified mRNA Encoding Amino Acid Sequence of Transcription Factor

Modified mRNA is synthesized with reference to a method described in the literature “Warren et al., Cell Stem Cell, 2010 Nov. 5; 7 (5): 618-30.” More specifically, mRNA is synthesized by in vitro transcription using a mixture of dNTPs {(dNTPs: 3-O-Me-m⁷G (5′) ppp (5′) G ARCA cap analog, 5-methylcytidine triphosphate, and pseudouridine triphosphate)} obtained by modifying template DNA encoding the amino acid sequence of the transcription factor required for induction of differentiation into the desired cell type.

### Generation of Sendai Virus Vector Encoding Transcription Factor Required for Induction of Differentiation into Desired Cell Type

In the present invention, the Sendai virus vector is used for the introduction of the transcription factor required for induction of differentiation into the desired cell type. Further, in order to express a mammalian (in particular, human) transcription factor, a Sendai virus vector capable of expressing a human transcription factor is preferably used. In particular, a mutant of a Sendai virus vector, such as an F protein-deficient mutant, has no infectivity, and hence is easy to handle (see Inoue et al., J Virol. 77.5:3238-3246, 2003).

### Method of Inducing Differentiation of Pluripotent Stem Cell into Desired Cell Type with High Efficiency

A single transcription factor or a cocktail of two or more transcription factors required for induction of differentiation into the desired cell type is prepared. The form of the transcription factor is a Sendai virus vector having incorporated therein a transcription factor (or a plurality of transcription factors).

### Use of Expression Vector

In a step of the method of the present invention, there may be used an expression vector known per se having introduced therein the gene for the compound having an action of substantially removing or reducing an expression amount of a POU5F1 protein (or the gene for the compound having an action of substantially removing or reducing H3K27me3 modification) and/or the transcription factor required for induction of differentiation into the desired cell type. An example of the expression vector to be used in the present invention may be a Sendai virus vector.

A method of introducing the expression vector into the pluripotent stem cell is not particularly limited, but examples thereof may include a lipofection method, a liposome method, an electroporation method, a calcium phosphate coprecipitation method, a diethylaminoethyl (DEAE)-dextran method, a microinjection method, and a gene gun method. A particularly preferred example is a lipofection method.

As another method, the gene for the compound having an action of substantially removing or reducing an expression amount of a POU5F1 protein (or the gene for the compound having an action of substantially removing or reducing H3K27me3 modification) may be converted to cationic siRNA by binding spermine, phosphospermine, or the like thereto. The cationic siRNA does not require a reagent for transfection.

### Compound Having Action of Substantially Removing or Reducing Expression Amount of POU5F1 Protein

The compound having an action of substantially removing or reducing an expression amount of a POU5F1 protein of the present invention is not particularly limited, but is, for example, siRNA against POU5F1, shRNA against POU5F1, an antisense strand of POU5F1, an antibody that specifically binds to the POU5F1 protein, or an inhibitor.

In addition, not only by using those compounds alone, but by using a plurality of kinds of compounds and/or a low-molecular-weight compound in combination, it is possible to efficiently “reduce the undifferentiation maintenance of a pluripotent stem cell (substantially remove or reduce an expression amount of a POU5F1 protein of a pluripotent stem cell).”

### Compound Having Action of Substantially Removing or Reducing H3K27me3 Modification

The compound having an action of substantially removing or reducing H3K27me3 modification of the present invention is not particularly limited, but is, for example, a demethylase (in particular, a demethylase having an action of removing a methyl group of H3K27me3), an antibody that specifically binds to H3K27me3, an antibody for Polycomb-group proteins (PcG proteins) having an H3K27me3 modification action, siRNA (in particular, cationic siRNA), or an inhibitor. The cationic siRNA does not require a reagent for transfection.

Examples of the compound may include low-molecular-weight compounds including, but not particularly limited to, histone deacetylase (HDAC) inhibitors, such as valproic acid. (JMJD3)

JMJD3 is known as a demethylase for H3K27me3 of a histone (mouse NP_001017426, human NP_001073893), and even in its full length (NP_001073893, SEQ ID NO: 3), has an action of substantially removing or reducing the H3K27me3 modification of pluripotent stem cells. The inventors of the present invention have confirmed that JMJD3c having the JmjC domain {SEQ ID NO: 4, catalytic domain: SEQ ID NO: 5 (amino acids 1376-1484)} has a stronger action of substantially removing or reducing H3K27me3 modification as compared to full-length JMJD3 (see WO 2017/073763 A1).

A preferred base sequence of JMJD3 is a base sequence set forth in SEQ ID NO: 6.

### Kind of Transcription Factor Required for Induction of Differentiation into Desired Cell Type

The form of the “transcription factor required for induction of differentiation into the desired cell type” to be used in the method of the present invention is not particularly limited as long as the transcription factor can be carried on the Sendai virus vector, but examples thereof may include, but not particularly limited to, nucleic acids, such as RNA and DNA, and synthetic nucleic acids.

In addition, in the method of the present invention, examples of the desired cell type may include skeletal muscles (skeletal muscle cells), the liver (liver cells), nerve cells (motor neurons and dopaminergic neurons), chondrocytes, bone cells, and hematopoietic cells.

As described in Examples below, in the method of the present invention, the number of kinds of transcription factors required for induction of differentiation into the desired cell type has been successfully reduced and/or high differentiation efficiency has been successfully achieved by carrying the transcription factor required for induction of differentiation into the desired cell type on the Sendai virus vector.

{Transcription Factor Required for Induction of Differentiation into Skeletal Muscle (in Particular, Cells Present in Skeletal Muscle)}

A method of inducing differentiation into a skeletal muscle is as described below.

A single transcription factor, or two or more transcription factors selected from the group consisting of MYOD1, NRF1, SALL4, ZIC1, KLF9, ZNF281, CTCF, HES1, HOXA2, TBX5, TP73, ERG, MAB21L3, PRDM1, NFIC, CTCFL, FOXP1, HEY1, PITX2, JUNB, KLF4, ESX1, TFAP2C, FOS, TFE3, FOSL1, GRHL2, TBX2, NFIB, and IRF4 are introduced into pluripotent stem cells. In particular, MYOD1 (base sequence: SEQ ID NO: 7, amino acid sequence: SEQ ID NO: 8) is preferably introduced alone into pluripotent stem cells.

### Transcription Factor Required for Induction of Differentiation into Nerve Cells

A method of inducing differentiation into nerve cells (in particular, motor neurons or dopaminergic neurons) is as described below.

A single transcription factor, or two, three, four, five, or six transcription factors selected from NEUROG1, NEUROG2, NEUROG3, NEUROD1, NEUROD2, and FOXA1 are introduced into human pluripotent stem cells.

For example, for the motor neurons (cholinergic and/or motor neurons), NEUROG3 (NGN3) (base sequence: SEQ ID NO: 9, amino acid sequence: SEQ ID NO: 10) is preferably introduced alone into pluripotent stem cells.

For example, for the dopaminergic neurons, FOXA1 (accession number: NM_004496) is preferably introduced alone into pluripotent stem cells.

{Transcription Factor Required for Induction of Differentiation into Liver (in Particular, Cells Present in Liver, i.e., Hepatoblasts)}

A method of inducing differentiation into the liver (in particular, the liver or the fetal liver) is as described below.

For the liver, only one transcription factor, or two or more transcription factors selected from HNF1A, TCF-1, SALL4, TGIF1, MAB21L3, ZIC1, EGFLAM, PITX2, HNF4A, NRF1, ZNF281, CTCFL, TP73, TFE3, DLX6, and TCF4 are introduced into human pluripotent stem cells.

For the fetal liver, only one transcription factor, or two or more transcription factors selected from HNF1A, TCF-1, SIX5, HNF4A, SIN3A, ID1, and HNF1A are introduced into human pluripotent stem cells.

In particular, the FOXA1 gene and HNF1A (base sequence: SEQ ID NO: 11, amino acid sequence: SEQ ID NO: 12) are preferably introduced into pluripotent stem cells.

### Transcription Factor Required for Induction of Differentiation into Hematopoietic Cells

A method of inducing differentiation into hematopoietic cells is as described below.

Only one transcription factor, or two, three, four, five, six, or seven transcription factors selected from CDYL2, ETS2, SPI1, OVOL2, CDX2, CEBPB, and SALL4 are introduced into human pluripotent stem cells.

In particular, SPI1 (base sequence: SEQ ID NO: 18, amino acid sequence: SEQ ID NO: 19) is preferably introduced into pluripotent stem cells.

### Method of Introducing Target Gene into Genome of Pluripotent Stem Cell

In a step of the method of the present invention, a method known per se may be used without any particular limitation as a method of introducing the gene for the compound having an action of substantially removing or reducing an expression amount of a POU5F1 protein (or the gene for the compound having an action of substantially removing or reducing H3K27me3 modification) into the genome of the pluripotent stem cell. There may be preferably used an expression cassette inserted between PiggyBac transposase recognition sequences (PB sequences) developed by Woltjen et al. (reference: Nature 458:766-770, 2009.), which is a mechanism by which a gene to be introduced is actively incorporated into pluripotent stem cells (in particular, the genome of human ES cells). The expression cassette is a system capable of efficiently establishing a genetically modified pluripotent stem cell line by introducing a drug selection cassette (see WO 2017/131238 A1).

### Method of Introducing Target Protein into Pluripotent Stem Cell

In a step of the method of the present invention, a method known per se may be used as a method of introducing (transfecting) the compound having an action of substantially removing or reducing an expression amount of a POU5F1 protein (or the compound having an action of substantially removing or reducing H3K27me3 modification) (in particular, a protein) into the pluripotent stem cell, and examples thereof may include: a method involving using a protein transfection reagent; a method involving using a fusion protein having added thereto a cell-penetrating peptide; and a microinjection method.

The “cell-penetrating peptide” in the present invention is a peptide having a property of migrating into a cell, more specifically a property of penetrating a cell membrane, still more specifically a property of penetrating a cell membrane or a nuclear membrane to penetrate into cytoplasm or a nucleus. The amino acid sequence of the peptide is not particularly limited, but examples thereof may include TAT (GRKKRRQRRRPQ: SEQ ID NO: 13), r8 {rrrrrrrr (D-form-R): SEQ ID NO: 14}, and MPG-8 (βAFLGWLGAWGTMGWSPKKKRK: SEQ ID NO: 15).

The target protein encompasses the compound having an action of substantially removing or reducing an expression amount of a POU5F1 protein (or the compound having an action of substantially removing or reducing H3K27me3 modification) (in particular, a protein).

### Gene Knockout Method

A gene knockout method is available as a method other than the foregoing. A “pluripotent stem cell in which a POU5F1 gene has been disrupted” may be generated by the gene knockout method. The “pluripotent stem cell in which a POU5F1 gene has been disrupted” means that normal expression of the POU5F1 gene is inhibited due to artificial modification of the sequence of a POU5F1 gene region, and as a result, the expression of POU5F1 is suppressed and a POU5F1 protein is not normally expressed.

In addition, the “whole” in “modification or deletion of part or the whole of the POU5S1 gene” refers to the protein-coding region of POU5F1 genomic DNA.

In addition, the “part” refers to a region that is part of the protein-coding region and that has a length required for inhibiting normal expression of the POU5F1 gene.

Further, the “modification” refers to modification of the base sequence of a target region in genomic DNA into another base sequence by substituting, deleting, inserting, and/or adding a single nucleotide or a plurality of nucleotides.

### Differentiation Induction Kit for Inducing Differentiation of Pluripotent Stem Cell into Desired Cell Type with High Efficiency

A differentiation induction kit for efficiently inducing differentiation of a pluripotent stem cell into a desired cell type of the present invention (hereinafter sometimes referred to as “kit of the present invention”) includes any one or more of the following items (1) to (5) in addition to a transcription factor required for induction of differentiation into the desired cell type and a Sendai virus vector.

(1) Pluripotent Stem Cell in Which Expression Amount of POU5F1 Protein has been Substantially Removed or Reduced and/or H3K27me3 Modification has been Substantially Removed or Reduced

A user can easily induce differentiation into the desired cell type by, as described above, introducing a Sendai virus vector containing a transcription factor required for induction of differentiation into the desired cell type into a pluripotent stem cell in which an expression amount of a POU5F1 protein has been substantially removed or reduced and/or H3K27me3 modification has been substantially removed or reduced.

In addition, such pluripotent stem cell encompasses a pluripotent stem cell having a gene construct inducible with doxycycline or the like inserted into the genome thereof so that a gene for a compound having an action of substantially removing or reducing an expression amount of a POU5F1 protein, a demethylase, or the like can be transiently forcibly expressed therein.

(2) Gene for Compound Having Action of Substantially Removing or Reducing Expression Amount of POU5F1 Protein and/or Demethylase Gene for Kit of the Present Invention

The user can easily generate the pluripotent stem cell in which an expression amount of a POU5F1 protein has been substantially removed or reduced and/or H3K27me3 modification has been substantially removed or reduced by adding a gene for a compound having an action of substantially removing or reducing an expression amount of a POU5F1 protein and/or a demethylase gene for a kit to a pluripotent stem cell.

Examples of anti-POU5F1 antibody gene may include, but not particularly limited to, commercially available antibody genes.

Examples of the demethylase gene for a kit may include, but not particularly limited to, mRNAs, DNAs, and proteins of demethylase genes (e.g., JMJD3c).

(3) Gene for Compound Having Action of Substantially Removing or Reducing Expression Amount of POU5F1 Protein and/or Demethylase Gene, and Sendai Virus Vector Containing Transcription Factor Required for Induction of Differentiation into Desired Cell Type for Kit of the Present Invention

The user can easily generate the pluripotent stem cell in which an expression amount of a POU5F1 protein has been substantially removed or reduced and/or H3K27me3 modification has been substantially removed or reduced, and further, can induce differentiation thereof into the desired cell type with high efficiency by adding a gene for a compound having an action of substantially removing or reducing an expression amount of a POU5F1 protein and/or a demethylase gene, and a Sendai virus vector containing a transcription factor required for induction of differentiation into the desired cell type for a kit to a pluripotent stem cell.

Those genes may exist on one gene, or on separate genes. When the genes are present on separate genes, the gene for the compound having an action of substantially removing or reducing an expression amount of a POU5F1 protein (or the demethylase gene) and the Sendai virus vector containing the transcription factor required for induction of differentiation into the desired cell type may be added to the pluripotent stem cell simultaneously or at separate times.

(4) Compound Having Action of Substantially Removing or Reducing Expression Amount of POU5F1 Protein and/or Demethylase for Kit of the Present Invention

The user can easily generate the pluripotent stem cell in which an expression amount of a POU5F1 protein has been substantially removed or reduced and/or H3K27me3 modification has been substantially removed or reduced by adding a compound having an action of substantially removing or reducing an expression amount of a POU5F1 protein and/or a demethylase for a kit to the pluripotent stem cell.

(5) Gene Construct carrying Gene for Compound having Action of Substantially Removing or Reducing Expression Amount of POU5F1 Protein and/or Demethylase Gene

The user can easily generate the pluripotent stem cell in which an expression amount of a POU5F1 protein has been substantially removed or reduced and/or H3K27me3 modification has been substantially removed or reduced by introducing a gene construct carrying a gene for a compound having an action of substantially removing or reducing an expression amount of a POU5F1 protein and/or a demethylase gene into the genome of a pluripotent stem cell.

The gene construct may contain a promoter sequence, a gene expression-enhancing sequence, a marker gene, a reporter sequence, a drug resistance gene, and the like as required in addition to the gene for the compound having an action of substantially removing or reducing an expression amount of a POU5F1 protein and/or the demethylase gene.

A method of the present invention may be exemplified by, but not particularly limited to, a method including any one of the following steps (1) to (8):
(1) a step of adding a gene for a compound having an action of substantially removing or reducing an expression amount of a POU5F1 protein and a Sendai virus vector containing a transcription factor required for induction of differentiation into the desired cell type to a pluripotent stem cell;
(2) a step of inserting a gene construct carrying a gene for a compound having an action of substantially removing or reducing an expression amount of a POU5F1 protein and a Sendai virus vector containing a transcription factor required for induction of differentiation into the desired cell type into the genome of a pluripotent stem cell;
(3) a step of adding a Sendai virus vector containing a transcription factor required for induction of differentiation into the desired cell type to a pluripotent stem cell in which an expression amount of a POU5F1 protein has been substantially removed or reduced;
(4) a step of adding a Sendai virus vector containing a transcription factor required for induction of differentiation into the desired cell type to a pluripotent stem cell in which a compound having an action of substantially removing or reducing an expression amount of a POU5F1 protein is forcibly expressed;
(5) a step of adding a compound having an action of substantially removing or reducing an expression amount of a POU5F1 protein and a Sendai virus vector containing a transcription factor required for induction of differentiation into the desired cell type to a pluripotent stem cell;
(6) a step of adding a Sendai virus vector containing a transcription factor required for induction of differentiation into the desired cell type to a pluripotent stem cell in which a POU5F1 gene has been disrupted;
(7) a step of adding a gene for a compound having an action of substantially removing or reducing an expression amount of a POU5F1 protein, a demethylase gene, and a Sendai virus vector containing a transcription factor required for induction of differentiation into the desired cell type to a pluripotent stem cell; and
(8) a step of adding a Sendai virus vector containing a transcription factor required for induction of differentiation into the desired cell type to a pluripotent stem cell in which an expression amount of a POU5F1 protein has been substantially removed or reduced and which has a histone in which H3K27me3 modification has been substantially removed or reduced.

In the method of the present invention, any one of the following pluripotent stem cells for differentiation into the desired cell type may also be used:
(1) a pluripotent stem cell for differentiation into the desired cell type, in which an expression amount of a POU5F1 protein has been substantially removed or reduced;
(2) a pluripotent stem cell for differentiation into the desired cell type, in which a gene for a compound having an action of substantially removing or reducing an expression amount of a POU5F1 protein is forcibly expressed;
(3) a pluripotent stem cell for differentiation into the desired cell type, which has a gene construct carrying a gene for a compound having an action of substantially removing or reducing an expression amount of a POU5F1 protein inserted into the genome thereof;
(4) a pluripotent stem cell for differentiation into the desired cell type, in which a POU5F1 gene has been disrupted; and
(5) a pluripotent stem cell for differentiation into the desired cell type, in which an expression amount of a POU5F1 protein has been substantially removed or reduced and which has a histone in which H3K27me3 modification has been substantially removed or reduced.

### EXAMPLES

The present invention is described below by way of Examples, but the present invention is by no means limited to Examples.

### Example 1

### Differentiation into Skeletal Muscles

In this Example, it was confirmed that, through the use of a temperature-sensitive Sendai virus vector expressing a human MYOD1 gene, hPSCs were able to be differentiated into skeletal muscles within 1 week. In this Example, it was also confirmed that the addition of siPOU5F1, which suppressed the expression of a pluripotency gene POU5F1 (also known as OCT4 or OCT3/4), further enhanced differentiation efficiency.

### Materials and Methods

### <Cell Culture>

An iPSC line derived from human adipose stem cells (iPSCs-ADSC) was obtained from System Biosciences (Palo Alto). Cells were maintained as undifferentiated pluripotent cells in accordance with a standard hPSC culture method involving using StemFit basic02 (Ajinomoto) supplemented with 100 ng/ml FGF2. The cells were cultured on a cell culture dish coated with lamin-511 (iMatrix-511, Nippi). For skeletal muscle differentiation, α-MEM (Thermo-Fisher) supplemented with 5% KSR (Thermo-Fisher) was used as a culture medium.

### <Sendai Virus Vector>

A temperature-sensitive Sendai virus vector expressing human MYOD1 (SeV18+hMYOD1/TS15AF) was custom-made by ID Pharma on contract. This Sendai virus vector is F protein-deficient, and hence is nontransmissible (Inoue et al., J Virol. 77.5:3238-3246, 2003). This Sendai virus vector is temperature-sensitive, and this virus functions at 33° C. and is inactivated at 37° C. (Ban et al., Proc Natl Acad Sci USA. 2011; 108(34): 14234-14239). A stock solution of the Sendai virus vector was diluted to 1×10⁴ cell-infecting units (CIU)/μl.

<Phosphospermine-bound siPOU5F1>

In order to suppress the expression of human POU5F1 in the hPSCs, small interfering RNAs (siRNAs) against human POU5F1 were designed and used. In order to introduce the siRNAs without using lipofection or any other cationic lipid-based transfection reagent, the siRNAs were bound with phosphospermine (Paris et al., Molecular Pharmaceutics 2012. 9:3464-3475). Sequences used are a human POU5F1 sense strand: 5′-GCCCGAAAGAGAAAGCGAAdT*dT-3′ (SEQ ID NO: 16) and a human POU5F1 antisense strand: 5′-UUCGCUUUCUCUUUCGGGCdCdT-3′ (SEQ ID NO: 17). The sense strand has 19 RNA bases and 2 DNA bases having added thereto 30 spermine molecules at the site indicated by *. The antisense strand has 19 RNA bases and 2 DNA bases. The sense strand and the antisense strand were annealed, and stored as a 100 μM stock solution.

### <Procedure>

(1) On Day 1, the human iPSCs (iPSCs-ADSC line) were plated in a 24-well plate or a 4-well plate at 1.0×10⁵ cells in 250 μl of the medium per well. The culture medium used was α-MEM (Thermo-Fisher) supplemented with 5% KSR (Thermo-Fisher). Immediately after the plating, the Sendai virus vector encoding the human MYOD1 gene was added to the cell culture medium at a multiplicity of infection (MOI) of 2.5. In this Example, 25 μl of a Sendai virus solution containing 2.5×10⁵ CIU was added to 250 μl of the medium containing 1.0×10⁵ cells. One hour after the addition of the Sendai virus vector, 1 ml of a culture solution was added to a total amount of 1.275 ml per well. The cells were cultured in a CO₂ incubator at 33° C., a permissive temperature for viral replication and gene expression. The culture medium (1 ml/well) was changed daily.
(2) On Day 2, 4 μl of phosphospermine-siPOU5F1 (100 μM stock solution) was added to 1 ml of the medium per well at a final concentration of 400 nM.

(3) On Day 4, the temperature of the CO₂ incubator was changed to 37° C., a non-permissive temperature for viral replication and gene expression.

(4) On Day 5, spindle-shaped cells serving as a clear indication of skeletal muscle differentiation were observed.

(5) On Day 6, the cells were fixed, and the efficiency of cell differentiation was evaluated using immunostaining. The immunostaining was performed by incubating the fixed cells overnight using an anti-myosin heavy chain (MHC) antibody (R&D systems) in 1:400 dilution. The cells were incubated for 1 hour using Alexa fluor 555 goat anti-mouse IgG as a secondary antibody in 1:200 dilution.

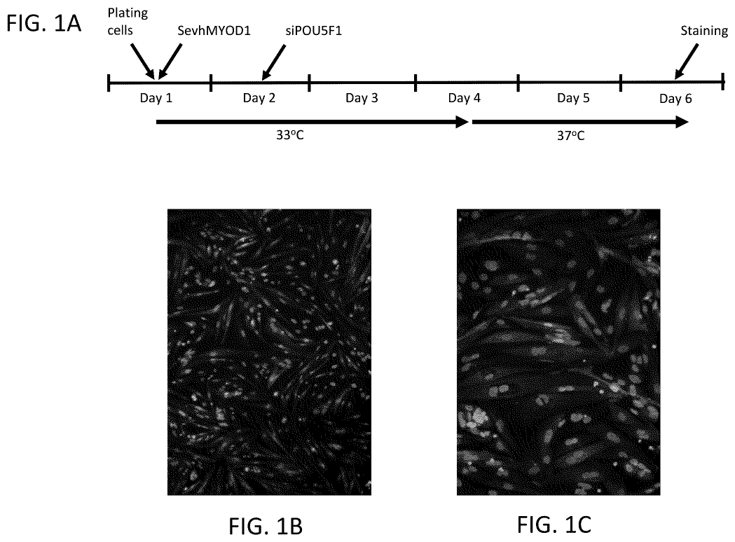

In FIG. 1A, a typical experimental procedure of the method of differentiating hPSCs into skeletal muscles according to the present invention is illustrated.

### Results

FIG. 1B is an example of a microscopic image (10× objective lens) of the cells immunostained with anti-myosin heavy chain (MHC) (red signal), which is specific for mature skeletal muscles. The cells were stained with DAPI (green signal) for visualizing the nuclei of all cells. An enlarged microscopic image (20× objective lens) of the cells is shown in FIG. 1C. Highly efficient formation of skeletal muscle cells was shown in visual inspection of the immunostaining result. By counting DAPI-positive cells and MHC-positive cells in a total of five images, it was found the average fraction (average differentiation efficiency) of the MHC-positive cells in the DAPI-positive cells was 84.7% (528 cells/623 cells). This average differentiation efficiency was the highest efficiency of skeletal muscle differentiation from hPSCs in the hitherto reported results. In addition, in the method of the present invention, an efficiency of skeletal muscle differentiation of 90% or more was observed.

As apparent from the above-mentioned results, the method of the present invention was able to achieve differentiation of hPSCs into skeletal muscle cells rapidly (5 days), efficiently and homogeneously (up to about 85% of MHC-positive skeletal muscle cells in all cells during culture), and simply (only one time of treatment with the Sendai virus vector, and only one time of treatment with phosphospermine-siPOU5F1). In Table 1, a comparison between the method of the present invention and related-art methods is shown.

TABLE 1

| | | Efficiency (% | |
|---|---|---|---|
| | Speed | MHC-positive | |
| Method | (days) | cells) | Effect |
| The present invention | 5 | ~85% | One time of Sendai virus |
| | | | infection; one time of |
| | | | phosphospermine- |
| | | | siPOU5F1 treatment. |
| | | | One kind of cell culture |
| | | | medium. |
| Synthetic mRNA | 5 | ~65% | Five times of trans- |
| cocktail (Akiyama et | | | fection with synthetic |
| al., Development. | | | mRNA cocktail. |
| 2016; 143: 3674- | | | Two kinds of |
| 3685) | | | cell culture media. |
| Successive changes | 20 | ~70% | Three kinds of cell |
| in cell culture | | | culture media. |
| environment | | | |
| (medium). AMSBIO | | | |
| (www.amsbio.com) | | | |
| Skeletal muscle | | | |
| differentiation kit | | | |

As apparent from the foregoing, as compared to the related-art methods, the method of the present invention is a method capable of achieving a skeletal muscle cell differentiation efficiency of from about 71% or more to about 90% or less, from about 73% or more to about 90% or less, from about 75% or more to about 90% or less, from about 78% or more to about 90% or less, from about 80% or more to about 90% or less, from about 82% or more to about 90% or less, from about 83% or more to about 90% or less, from about 84% or more to about 90% or less, from about 85% to about 90% or less, about 85%, about 90%, or about 90% or more with one kind of medium and through one time of transcription factor introduction. Further, the method of the present invention is a method capable of achieving the above-mentioned differentiation efficiency within 12 days, within 10 days, within 9 days, within 8 days, within 7 days, within 6 days, or within 5 days.

### Example 2

### Differentiation into Motor Neurons

In this Example, it was confirmed that, through the use of a temperature-sensitive Sendai virus vector expressing a human NGN3 gene, hPSCs were able to be differentiated into cholinergic and/or motor neurons within 1 week.

### Materials and Methods

### <Cell Culture>

An iPSC line derived from human adipose stem cells (iPSCs-ADSC) was obtained from System Biosciences (Palo Alto). Cells were maintained as undifferentiated pluripotent cells in accordance with a standard hPSC culture method involving using StemFit basic02 (Ajinomoto) supplemented with 100 ng/ml FGF2. The cells were cultured on a cell culture dish coated with lamin-511 (iMatrix-511, Nippi). For motor neuron differentiation, a 1:1 mixture of DMEM/F12 HAM and Neurobasal medium was used as a cell culture medium. The medium was supplemented with 1×N2/B27, dorsomorphin (final concentration: 3.3 μM), SB431542 (final concentration: 3.3 μM), and forskolin (final concentration: 3.3 μM).

### <Sendai Virus Vector>

A temperature-sensitive Sendai virus vector expressing human NGN3 (SeV18+hNGN3/TS15ΔF) was custom-made by ID Pharma on contract. This Sendai virus vector is F protein-deficient, and hence is nontransmissible. This Sendai virus vector is temperature-sensitive, and this virus functions at 33° C. and is inactivated at 37° C. A stock solution of the Sendai virus vector was diluted to 1×10⁴ CIU/μl.

### <Procedure>

(1) On Day 1, the human iPSCs (iPSCs-ADSC line) were plated in a 24-well plate or a 4-well plate at 1.0×10⁵ cells in 250 μl of the medium per well. The culture medium is as described above (1:1 mixture of DMEM/F12 HAM and Neurobasal medium supplemented with 1×N2/B27, dorsomorphin, SB431542, and forskolin). Immediately after the plating, the Sendai virus vector encoding the human NGN3 gene was added to the cell culture medium at a multiplicity of infection (MOI) of 2.5. In this Example, 25 μl of a Sendai virus solution containing 2.5×10⁵ CIU was added to 250 μl of the medium containing 1.0×10⁵ cells. One hour after the addition of the Sendai virus vector, 1 ml of a culture solution was added to a total amount of 1.275 ml per well. The cells were cultured in a CO₂ incubator at 33° C., a permissive temperature for viral replication and gene expression. The culture medium (1 ml/well) was changed daily.
(2) On Day 3, the temperature of the CO₂ incubator was changed to 37° C., a non-permissive temperature for viral replication and gene expression.
(3) As an optional procedure, on Day 4, the cells were passaged, and cultured on an ornithine/laminin-coated glass coverslip so that differentiated neurons were easily visible under a microscope.
(4) On Day 6, the cells were fixed, and the efficiency of cell differentiation was evaluated using immunostaining. The immunostaining was performed by incubating the fixed cells overnight using an anti-tubulin β3 (TUBB3) antibody or anti-choline acetyltransferase (ChAT) antibody.

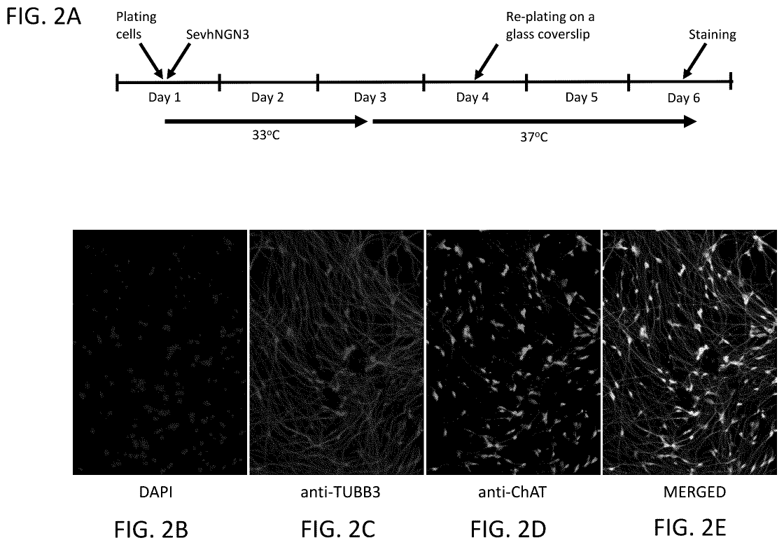

In FIG. 2A, a typical experimental procedure of the method of differentiating hPSCs into neurons according to the present invention is illustrated.

### Results

FIG. 2B to FIG. 2D are examples of microscopic images (20× objective lens) of the immunostained cells. FIG. 2B is an image of the cells stained with DAPI (blue signal) for visualizing the nuclei of all cells. FIG. 2C is an image of the cells immunostained with anti-β3-tubulin (TUBB3) antibody (red signal), which is specific for mature neurons. FIG. 2D is an image of the cells immunostained with anti-choline acetyltransferase (ChAT) antibody (green signal), which is specific for motor neurons. FIG. 2E is a synthetic image of FIG. 2C and FIG. 2D. By counting DAPI-positive cells and TUBB3-positive cells in a total of five images, it was found that the average fraction of the TUBB3-positive cells in the DAPI-positive cells (average differentiation efficiency) was 89.5% (205 cells/229 cells). This result of average differentiation efficiency shows that the method of the present invention can produce up to about 90% of neurons from hPSCs. By counting ChAT-positive cells and TUBB3-positive cells in a total of five images, it was found that the average fraction of the ChAT-positive cells in the TUBB3-positive cells was 93.2% (191 cells/205 cells). This result shows that most of the neurons produced using the combination of the Sendai virus vector expressing NGN3 and the differentiation medium are motor neurons. The efficiency was confirmed to be the highest efficiency of motor neuron differentiation from hPSCs in the hitherto reported results.

As apparent from the above-mentioned results, the method of the present invention was able to achieve differentiation of hPSCs into motor neurons rapidly (5 days), efficiently and homogeneously (up to about 90% of TUBB3-positive neurons in all cells during culture), and simply (only one time of treatment with the Sendai virus vector). In Table 2, a comparison between the method of the present invention and related-art methods is shown.

TABLE 2

| | | Efficiency | |
|---|---|---|---|
| | | (% TUBB3- | |
| | Speed | positive | |
| Method | (days) | cells) | Effect |
| The present invention | 5 | ~90% | One time of Sendai virus |
| | | | infection. |
| | | | One kind of cell culture |
| | | | medium. |
| Synthetic mRNA | 5 | ~90% | Two to four times of |
| cocktail (Goparaju et | | | transfection with |
| al., Scientific Reports. | | | synthetic mRNA cocktail. |
| 2017; 7: 42367) | | | Two kinds of cell culture |
| | | | media. |
| Successive changes in | 10 | ~75% | Five kinds of cell culture |
| cell culture | | | media. |
| environment (medium) | | | |
| (Chambers et al. 2012. | | | |
| Nature Biotech 7: 715.) | | | |

As apparent from the foregoing, as compared to the related-art methods, the method of the present invention is a method capable of achieving a motor neuron differentiation efficiency of from about 76% or more to about 90% or less, from about 78% or more to about 90% or less, from about 80% or more to about 90% or less, from about 82% or more to about 90% or less, from about 84% or more to about 90% or less, from about 86% or more to about 90% or less, from about 88% or more to about 90% or less, or about 90% with one kind of medium and through one time of transcription factor introduction. Further, the method of the present invention is a method capable of achieving the above-mentioned differentiation efficiency within 12 days, within 10 days, within 9 days, within 8 days, within 7 days, within 6 days, or within 5 days.

### Example 3

### Differentiation into Liver Cells

In this Example, it was confirmed that a temperature-sensitive Sendai virus vector expressing a human FOXA1 gene and/or an HNF1A gene was able to differentiate hPSCs into liver cells.

### Materials and Methods

### <Cell Culture>

An iPSC line derived from human adipose stem cells (iPSCs-ADSC) was obtained from System Biosciences (Palo Alto). Cells were maintained as undifferentiated pluripotent cells in accordance with a standard hPSC culture method involving using StemFit basic02 (Ajinomoto) supplemented with 100 ng/ml FGF2. The cells were cultured on a cell culture dish coated with lamin-511 (iMatrix-511, Nippi). For liver cell differentiation, media described in the literature (Hay et al., Stem Cells. 2008, 26:894-902 and Kajiwara et al., Proc Natl Acad Sci USA. 2012, 109: 14716) were used. Both cases in the literature took 18 days for differentiation, and used four kinds of cell culture media, i.e., priming medium A (1 day), priming medium B (3 days), differentiation medium (7 days), and maturation medium (7 days). On the other hand, as described below, the method of the present invention took only 8 days for differentiation, and used only two kinds of differentiation media, i.e., differentiation medium (5 days) and maturation medium (1 day).

### <Sendai Virus Vector>

A temperature-sensitive Sendai virus vector expressing human HNF1A (SeV18+hHNF1A/TS15AF) and a temperature-sensitive Sendai virus vector expressing FOXA1 (SeV18+hFOXA1/TS15AF) were custom-made by ID Pharma on contract. This Sendai virus vector is F protein-deficient, and hence is nontransmissible. This Sendai virus vector is temperature-sensitive, and this virus functions at 33° C. and is inactivated at 37° C. A stock solution of the Sendai virus vector was diluted to 1×10⁴ CIU/μl.

<Phosphospermine-bound siPOU5F1>

In order to suppress the expression of human POU5F1 in the hPSCs, small interfering RNAs (siRNAs) against human POU5F1 were designed and used. In order to introduce the siRNAs without using lipofection or any other cationic lipid-based transfection reagent, the siRNAs were bound with phosphospermine (Paris et al., Molecular Pharmaceutics 2012. 9:3464-3475). Sequences used are a human POU5F1 sense strand: 5′-GCCCGAAAGAGAAAGCGAAdT*dT-3′ (SEQ ID NO: 16) and a human POU5F1 antisense strand: 5′-UUCGCUUUCUCUUUCGGGCdCdT-3′ (SEQ ID NO: 17). The sense strand has 19 RNA bases and 2 DNA bases having added thereto 30 spermine molecules at the site indicated by *. The antisense strand has 19 RNA bases and 2 DNA bases. The sense strand and the antisense strand were annealed, and stored as a 100 μM stock solution.

### <Procedure>

(1) On Day 1, the human iPSCs (iPSCs-ADSC line) were plated in a 24-well plate or a 4-well plate at 1.0×10⁵ cells in 250 μl of the medium per well. The cell culture medium used was a ROCK inhibitor (Y27632)-containing StemFit (registered trademark in Japan) Basic02 (Ajinomoto). Immediately after the plating, a mixture of the Sendai virus vector encoding the human HNF1A gene (25 μl of a Sendai virus solution containing 2.5×10⁵ CIU) and the FOXA1 gene (25 μl of a Sendai virus solution containing 2.5×10⁵ CIU) was added to the cell culture medium. One hour after the addition of the Sendai virus vector, 1 ml of a culture solution was added to a total amount of 1.30 ml per well. The cells were cultured in a CO₂ incubator at 33° C., a permissive temperature for viral replication and gene expression. The culture medium (1 ml/well) was changed daily.
(2) On Day 2, 4 μl of phosphospermine-siPOU5F1 (100 μM stock solution) was added to 1 ml of the medium per well at a final concentration of 400 nM.
(3) On Day 2, the cell culture medium was replaced with a differentiation medium containing 1% DMSO.
(4) On Day 5, the temperature of the CO₂ incubator was changed to 37° C., a non-permissive temperature for viral replication and gene expression.
(5) On Day 7, the cell culture medium was replaced with a maturation medium containing L15 medium supplemented with 20 ng/ml hepatocyte growth factor (HGF), 20 ng/ml oncostatin M (OSM), 1 μM dexamethasone, 10 μM SB431542, 10 μM ROCK inhibitor, and 0.1 mg/ml ascorbic acid.
(6) On Day 8, the cells were fixed, and the efficiency of cell differentiation was evaluated using immunostaining. The immunostaining was performed by incubating the fixed cells using an anti-albumin antibody.

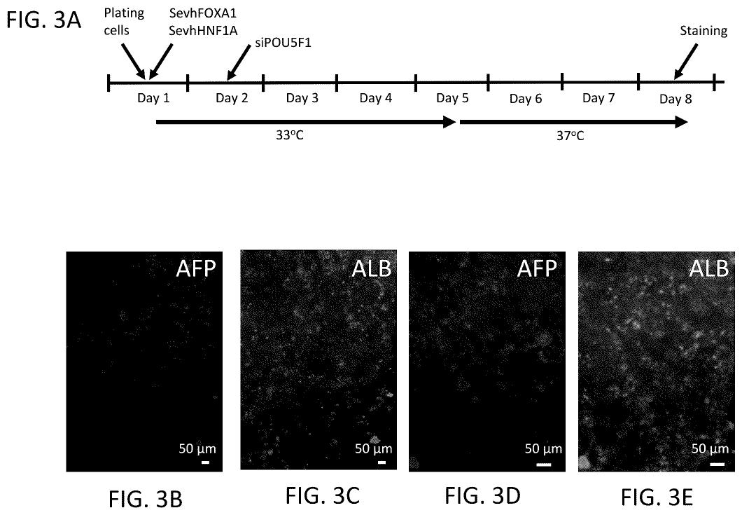

In FIG. 3A, a typical experimental procedure of the method of differentiating hPSCs into liver cells according to the present invention is illustrated.

### Results

It was confirmed that the method of the present invention was able to achieve differentiation of hPSCs into liver cells rapidly (8 days), efficiently and homogeneously, and simply (only one time of treatment with the Sendai virus vector, and only one time of treatment with phosphospermine-siPOU5F1) with the two kinds of media (see FIG. 3B to FIG. 3E).

### Example 4

### Differentiation into Hematopoietic Cells

In this Example, it is confirmed that a temperature-sensitive Sendai virus vector expressing a human SPI1 gene can differentiate hPSCs into hematopoietic cells.

### Materials and Methods

### <Cell Culture>

An iPSC line derived from human adipose stem cells (iPSCs-ADSC) was obtained from System Biosciences (Palo Alto). Cells were maintained as undifferentiated pluripotent cells in accordance with a standard hPSC culture method involving using StemFit basic02 (Ajinomoto) supplemented with 100 ng/ml FGF2. The cells were cultured on a cell culture dish coated with lamin-511 (iMatrix-511, Nippi). For hematopoietic cell differentiation, α-MEM (Thermo-Fisher) supplemented with 5% KSR (Thermo-Fisher) was used as a culture medium.

### <Sendai Virus Vector>

A temperature-sensitive Sendai virus vector expressing human SPI1 (SeV18+hSPI1/TS15AF) was custom-made by ID Pharma on contract. This Sendai virus vector is F protein-deficient, and hence is nontransmissible. This Sendai virus vector is temperature-sensitive, and this virus functions at 33° C. and is inactivated at 37° C. A stock solution of the Sendai virus vector was diluted to 1×10⁴ CIU/μl.

<Phosphospermine-bound siPOU5F1>

In order to suppress the expression of human POU5F1 in the hPSCs, small interfering RNAs (siRNAs) against human POU5F1 were designed and used. In order to introduce the siRNAs without using lipofection or any other cationic lipid-based transfection reagent, the siRNAs were bound with phosphospermine (Paris et al., Molecular Pharmaceutics 2012. 9:3464-3475). Sequences used are a human POU5F1 sense strand: 5′-GCCCGAAAGAGAAAGCGAAdT*dT-3′ (SEQ ID NO: 16) and a human POU5F1 antisense strand: 5′-UUCGCUUUCUCUUUCGGGCdCdT-3′ (SEQ ID NO: 17). The sense strand has 19 RNA bases and 2 DNA bases having added thereto 30 spermine molecules at the site indicated by *. The antisense strand has 19 RNA bases and 2 DNA bases. The sense strand and the antisense strand were annealed, and stored as a 100 μM stock solution.

### <Procedure>

(1) On Day 1, the human iPSCs (iPSCs-ADSC line) are plated in a 24-well plate or a 4-well plate at 1.0×10⁵ cells in 250 μl of the medium per well. The cell culture medium used is α-MEM (Thermo-Fisher) supplemented with 5% KSR (Thermo-Fisher). Immediately after the plating, the Sendai virus vector encoding the human SPI1 gene is added to the cell culture medium at a multiplicity of infection (MOI) of 2.5. In this Example, 25 μl of a Sendai virus solution containing 2.5×10⁵ CIU is added to 250 μl of the medium containing 1.0×10⁵ cells. One hour after the addition of the Sendai virus vector, 1 ml of a culture solution is added to a total amount of 1.275 ml per well. The cells are cultured in a CO₂ incubator at 33° C., a permissive temperature for viral replication and gene expression. The culture medium (1 ml/well) is changed daily.
(2) On Day 2, 4 μl of phosphospermine-siPOU5F1 (100 μM stock solution) is added to 1 ml of the medium per well at a final concentration of 400 nM.
(3) On Day 4, the temperature of the CO₂ incubator is changed to 37° C., a non-permissive temperature for viral replication and gene expression.
(4) On Day 6, the cells are fixed, and the efficiency of cell differentiation is evaluated using immunostaining. The immunostaining is performed by incubating the fixed cells using an anti-CD45 antibody.

### Results

It can be confirmed that the method of the present invention can achieve differentiation of hPSCs into hematopoietic CD45-positive cells rapidly (5 days), efficiently and homogeneously, and simply (only one time of treatment with the Sendai virus vector, and only one time of treatment with phosphospermine-siPOU5F1).

### Example 5

### Differentiation into Dopaminergic Neurons

In this Example, it is confirmed that a temperature-sensitive Sendai virus vector expressing a human FOXA1 gene can differentiate hPSCs into dopaminergic neurons within 1 week.

### Materials and Methods

### <Cell Culture>

An iPSC line derived from human adipose stem cells (iPSCs-ADSC) was obtained from System Biosciences (Palo Alto). Cells were maintained as undifferentiated pluripotent cells in accordance with a standard hPSC culture method involving using StemFit basic02 (Ajinomoto) supplemented with 100 ng/ml FGF2. The cells were cultured on a cell culture dish coated with lamin-511 (iMatrix-511, Nippi).

For dopaminergic neuron differentiation, a 1:1 mixture of DMEM/F12 HAM and Neurobasal medium was used as a cell culture medium. The medium was supplemented with 1×N2/B27 (without vitamin A and retinoic acid), BDNF (20 ng/ml), GDNF (20 ng/ml), TGF-β3 (1 ng/ml), ascorbic acid (final concentration: 0.2 mM), and cAMP (final concentration: 0.5 mM).

### <Sendai Virus Vector>

A temperature-sensitive Sendai virus vector expressing human FOXA1 (SeV18+hFOXA1/TS15ΔF) was custom-made by ID Pharma on contract. This Sendai virus vector is F protein-deficient, and hence is nontransmissible. This Sendai virus vector is temperature-sensitive, and this virus functions at 33° C. and is inactivated at 37° C. A stock solution of the Sendai virus vector was diluted to 1×10⁴ CIU/μl.

### <Procedure>

(1) On Day 1, the human iPSCs (iPSCs-ADSC line) are plated in a 24-well plate or a 4-well plate at 1.0×10⁵ cells in 250 μl of the medium per well. The above-mentioned culture medium is used (1:1 mixture of DMEM/F12 HAM and Neurobasal medium supplemented with 1×N2/27, BDNF, GDNF, TGF-β3, ascorbic acid, and cAMP). Immediately after the plating, the Sendai virus vector encoding the human FOXA1 gene is added to the cell culture medium at a multiplicity of infection (MOI) of 2.5. In this Example, 25 μl of a Sendai virus solution containing 2.5×10⁵ CIU is added to 250 μl of the medium containing 1.0×10⁵ cells. One hour after the addition of the Sendai virus vector, 1 ml of a culture solution is added to a total amount of 1.275 ml per well. The cells are cultured in a CO₂ incubator at 33° C., a permissive temperature for viral replication and gene expression. The culture medium (1 ml/well) is changed daily.
(2) On Day 3, the temperature of the CO₂ incubator is changed to 37° C., a non-permissive temperature for viral replication and gene expression.
(3) As an optional procedure, on Day 4, the cells are passaged, and cultured on an ornithine/laminin-coated glass coverslip so that differentiated neurons are easily visible under a microscope.
(4) On Day 6, the cells are fixed, and the efficiency of cell differentiation is evaluated using immunostaining. The immunostaining is performed by incubating the fixed cells overnight using an anti-tubulin β3 (TUBB3) antibody and tyrosine hydroxylase (TH).

### Results

It is confirmed that the method of the present invention can achieve differentiation of hPSCs into TH-positive dopaminergic neurons rapidly (5 days), efficiently and homogeneously, and simply (only one time of treatment with the Sendai virus vector).

According to the present invention, the method of inducing differentiation of a pluripotent stem cell into a desired cell type within a short period of time and with high efficiency can be provided.

1-15. (canceled)

16. A method of differentiating a pluripotent stem cell into a hematopoietic cell, comprising: a) adding a Sendai virus vector encoding a transcription factor SPI1 to a pluripotent stem cell in a cell culture medium; b) culturing the pluripotent stem cell to differentiate the pluripotent stem cell into the hematopoietic cell.

17. The method according to claim 16, further comprising adding a nucleic acid encoding a compound having an action of substantially removing or reducing an expression amount of POU5F1 protein to the pluripotent stem cell.

18. The method according to claim 17, wherein the nucleic acid encoding the compound having an action of substantially removing or reducing an expression amount of a POU5F1 protein encodes siRNA against POU5F1.

19. The method according to claim 16, wherein the Sendai virus vector is temperature-sensitive.

20. The method according to claim 17, wherein the Sendai virus vector is temperature-sensitive.

21. The method according to claim 16, wherein the adding the Sendai virus vector encoding the transcription factor SPI1 to the pluripotent stem cell in the cell culture medium is performed once.

22. The method according to claim 17, wherein the adding the Sendai virus vector encoding the transcription factor SPI1 to the pluripotent stem cell in the cell culture medium is performed once.


SEQUENCE LISTING (19 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - DNA, 1083 bp, Homo sapiens
  source 1..1083: mol_type = genomic DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 3 - protein, 1682 aa, Homo sapiens
MHRAVDPPGARAAREAFALGGLSCAGAWSSCPPHPPPRSAWLPGGRCSASIGQPPLPAPLPPSHGSSSGHPSKPYYAPGAPTPRPLHGKLESLHGCVQALLREPAQPGLWEQLGQLYESEHDSEEATRCYHSALRYGGSFAELGPRIGRLQQAQLWNFHTGSCQHRAKVLPPLEQVWNLLHLEHKRNYGAKRGGPPVKRAAEPPVVQPVPPAALSGPSGEEGLSPGGKRRRGCNSEQTGLPPGLPLPPPPLPPPPPPPPPPPPPLPGLATSPPFQLTKPGLWSTLHGDAWGPERKGSAPPERQEQRHSLPHPYPYPAPAYTAHPPGHRLVPAAPPGPGPRPPGAESHGCLPATRPPGSDLRESRVQRSRMDSSVSPAATTACVPYAPSRPPGLPGTTTSSSSSSSSNTGLRGVEPNPGIPGADHYQTPALEVSHHGRLGPSAHSSRKPFLGAPAATPHLSLPPGPSSPPPPPCPRLLRPPPPPAWLKGPACRAAREDGEILEELFFGTEGPPRPAPPPLPHREGFLGPPASRFSVGTQDSHTPPTPPTPTTSSSNSNSGSHSSSPAGPVSFPPPPYLARSIDPLPRPPSPAQNPQDPPLVPLTLALPPAPPSSCHQNTSGSFRRPESPRPRVSFPKTPEVGPGPPPGPLSKAPQPVPPGVGELPARGPRLFDFPPTPLEDQFEEPAEFKILPDGLANIMKMLDESIRKEEEQQQHEAGVAPQPPLKEPFASLQSPFPTDTAPTTTAPAVAVTTTTTTTTTTTATQEEEKKPPPALPPPPPLAKFPPPSQPQPPPPPPPSPASLLKSLASVLEGQKYCYRGTGAAVSTRPGPLPTTQYSPGPPSGATALPPTSAAPSAQGSPQPSASSSSQFSTSGGPWARERRAGEEPVPGPMTPTQPPPPLSLPPARSESEVLEEISRACETLVERVGRSATDPADPVDTAEPADSGTERLLPPAQAKEEAGGVAAVSGSCKRRQKEHQKEHRRHRRACKDSVGRRPREGRAKAKAKVPKEKSRRVLGNLDLQSEEIQGREKSRPDLGGASKAKPPTAPAPPSAPAPSAQPTPPSASVPGKKAREEAPGPPGVSRADMLKLRSLSEGPPKELKIRLIKVESGDKETFIASEVEERRLRMADLTISHCAADVVRASRNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEEKESEDEESEEPDSTTGTPPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLVRARRARGQRRRALGQAAGTGFGSPAAPFPEPPPAFSPQAPASTSR

SEQ ID NO: 4 - protein, 659 aa, Homo sapiens
QSEEIQGREKSRPDLGGASKAKPPTAPAPPSAPAPSAQPTPPSASVPGKKAREEAPGPPGVSRADMLKLRSLSEGPPKELKIRLIKVESGDKETFIASEVEERRLRMADLTISHCAADVVRASRNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEEKESEDEESEEPDSTTGTPPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLVRARRARGQRRRALGQAAGTGFGSPAAPFPEPPPAFSPQAPASTSR

SEQ ID NO: 5 - protein, 109 aa, Homo sapiens
QLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNV

SEQ ID NO: 6 - DNA, 2541 bp, Homo sapiens
  source 1..2541: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggcttagccaccatgtacccatacgatgttccagattacgctcctaagaaaaagaggaaggtgcagagcgaggagatccagggtcgtgagaagtcccggcccgatcttggcggggcctccaaggccaagccacccacagctccagcccctccatcagctcctgcaccttctgcccagcccacacccccgtcagcctctgtccctggaaagaaggctcgggaggaagccccagggccaccgggtgtcagccgggccgacatgctgaagctgcgctcacttagtgaggggccccccaaggagctgaagatccggctcatcaaggtagagagtggtgacaaggagacctttatcgcctctgaggtggaagagcggcggctgcgcatggcagacctcaccatcagccactgtgctgctgacgtcgtgcgcgccagcaggaatgccaaggtgaaagggaagtttcgagagtcctacctttcccctgcccagtctgtgaaaccgaagatcaacactgaggagaagctgccccgggaaaaactcaacccccctacacccagcatctatctggagagcaaacgggatgccttctcacctgtcctgctgcagttctgtacagaccctcgaaatcccatcacagtgatccggggcctggcgggctccctgcggctcaacttgggcctcttctccaccaagaccctggtggaagcgagtggcgaacacaccgtggaagttcgcacccaggtgcagcagccctcagatgagaactgggatctgacaggcactcggcagatctggccttgtgagagctcccgttcccacaccaccattgccaagtacgcacagtaccaggcctcatccttccaggagtctctgcaggaggagaaggagagtgaggatgaggagtcagaggagccagacagcaccactggaacccctcctagcagcgcaccagacccgaagaaccatcacatcatcaagtttggcaccaacatcgacttgtctgatgctaagcggtggaagccccagctgcaggagctgctgaagctgcccgccttcatgcgggtaacatccacgggcaacatgctgagccacgtgggccacaccatcctgggcatgaacacggtgcagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtggggcccctcaccgcctatcagtaccagctggccctggaacgatacgagtggaatgaggtgaagaacgtcaaatccatcgtgcccatgattcacgtgtcatggaacgtggctcgcacggtcaaaatcagcgaccccgacttgttcaagatgatcaagttctgcctgctgcagtccatgaagcactgccaggtgcaacgcgagagcctggtgcgggcagggaagaaaatcgcttaccagggccgtgtcaaggacgagccagcctactactgcaacgagtgcgatgtggaggtgtttaacatcctgttcgtgacaagtgagaatggcagccgcaacacgtacctggtacactgcgagggctgtgcccggcgccgcagcgcaggcctgcagggcgtggtggtgctggagcagtaccgcactgaggagctggctcaggcctacgacgccttcacgctggtgagggcccggcgggcgcgcgggcagcggaggagggcactggggcaggctgcagggacgggcttcgggagcccggccgcgcctttccctgagcccccgccggctttctccccccaggccccagccagcacgtcgcgatgaacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 1574 bp, Homo sapiens
  source 1..1574: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccgcggccgcccccttcaccgctagggataacagggtaatagaaggagccgccaccatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgagtttcctgtgaacaattgctcctctcttaaggtagcaaagggtgggcgcgccgacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 9 - DNA, 1153 bp, Homo sapiens
  source 1..1153: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggcttcaccatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgagacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 11 - DNA, 2414 bp, Homo sapiens
  source 1..2414: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccaccatggtttctaaactgagccagctgcagacggagctcctggcggccctgctggagtcagggctgagcaaagaggcactgctccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacaggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagttgtgagcggccgcacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 632 aa, Homo sapiens
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQL

SEQ ID NO: 13 - protein, 12 aa, Human immunodeficiency virus
GRKKRRQRRRPQ

SEQ ID NO: 14 - protein, 8 aa, synthetic construct
  REGION 1..8: note = r8
RRRRRRRR

SEQ ID NO: 15 - protein, 21 aa, synthetic construct
  REGION 1..21: note = MPG-8
  SITE 1: note = MISC_FEATURE - Xaa stands for bAla.
XFLGWLGAWGTMGWSPKKKRK

SEQ ID NO: 16 - DNA, 20 bp, synthetic construct
  misc_feature 1..20: note = siPOU5F1 sense strand
  source 1..20: mol_type = other DNA
  misc_feature 1..19: note = RNA
  misc_feature 20: note = DNA
  misc_feature 20: note = The 3' side of the thymine at position 20 is bound to 30 spermines. The 5' side of the 30 spermines is bounded to deoxythymine.
  modified_base 20: mod_base = OTHER; note = deoxythymidine
gcccgaaagagaaagcgaat

SEQ ID NO: 17 - DNA, 21 bp, synthetic construct
  misc_feature 1..21: note = siPOU5F1 anti-sense strand
  source 1..21: mol_type = other DNA
  misc_feature 20..21: note = DNA
  misc_feature 1..19: note = RNA
  modified_base 20: mod_base = OTHER; note = deoxycytidine
  modified_base 21: mod_base = OTHER; note = deoxythymidine
ttcgctttctctttcgggcct

SEQ ID NO: 18 - DNA, 816 bp, Homo sapiens
  source 1..816: mol_type = genomic DNA
atgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactga

SEQ ID NO: 19 - protein, 271 aa, Homo sapiens
MLQACKMEGFPLVPPQPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH